(19) United States
(12) Patent Application Publication
(10) Pub. No.: US 2021/0128493 A1
(43) Pub. Date: May 6, 2021

(54) Pet Food Supplement

(71) Applicants: Mina El Meniawy, Stoney Creek (CA); Robert Nashat, Stoney Creek (CA)

(72) Inventors: Mina El Meniawy, Stoney Creek (CA); Robert Nashat, Stoney Creek (CA)

(21) Appl. No.: 16/673,117

(22) Filed: Nov. 4, 2019

(51) Int. Cl. A61K 31/12; A61K 31/7008; A61K 31/737; A61K 31/10; A61K 31/728; A61K 31/51; A61K 31/341; A61K 31/355; A61K 33/32; A61K 33/30; A61K 36/03; A61K 36/87; A61K 31/4415; A61K 31/714; A61K 31/07; A61K 31/05; A61K 31/122; A61K 9/00

(52) U.S. Cl. CPC A61K 31/12; A61K 9/0056; A61K 31/737; A61K 31/10; A61K 31/728; A61K 31/51; A61K 31/341; A61K 31/355; A61K 33/32; A61K 33/30; A61K 36/03; A61K 36/87; A61K 31/4415; A61K 31/714; A61K 31/07; A61K 31/05; A61K 31/122; A61K 31/7008

(57) ABSTRACT

A pet food supplement is provided. The pet food supplement includes the following therapeutic components: curcumin bioavailability, joint preserving and building, fat soluble vitamin absorption, immune system preserving and building, and cancer prevention. One embodiment of the pet food supplement includes turmeric, BIOPERINE®, glucosamine, Chondroitin Sulfate, Methylsulfonylmethane, Sodium Hyaluronate, Vitamin B1, Vitamin C, Vitamin E, Manganese, Zinc, kelp, grape seed extract, Vitamin B6, Vitamin B9, Vitamin B12, the Vitamin C, Vitamin A, Vitamin D, Vitamin K, Omega-3, and Omega-6. Each of these ingredients is premixed and orally delivered to the pet in the form of a powder, paste or segment having a size and weight or other dosage measuring feature adjusted in accordance with a weight of the pet.

### BACKGROUND OF THE INVENTION

The present invention relates to a pet food supplement. More specifically, the present invention relates to a pet food supplement for mixing with a food of the pet having selected nutrition, vitamins, and minerals. Moreover, the present invention relates to therapeutic support for joints, immunity, energy, digestive health, skin and coat protection, sight, and liver health.

Pets such as dogs and cats are most often fed with processed pet food products. These processed pet food products may give rise to health problems of the pet due to the lack of specific nutrients, alone or in combination. These health problems which are associated with the consumption of processed pet food products include diarrhea, congestion, flatulence, lack of appetite, vomiting, loss of hair, unspecific itching of the skin, or skin diseases.

The nutrition and health of dogs and cats is one of the most important aspects of pet care. Many dog owners have a difficult time determining if their dog is receiving a well-balanced and healthy diet. For example, a conventional dog food includes a combination of water, proteins, carbohydrates, fats, vitamins, minerals and amino acids. However, the dog food typically lacks minerals, vitamins, and other supplements directed to enhanced joint health, digestive health, improved skin and coat protection, and liver health. Therefore, there exists a need for a pet food supplement that provides pets with the daily nutritional requirements to ensure good health.

In view of the above concerns, it is desirable to provide an embodiment of the pet food supplement that forms a powder that is selectively dosed and mixed with conventional pet food. Particularly, it is desirable to provide a pet food supplement that directed to complete pet nutrition.

In light of the food compositions disclosed in the known art, it is submitted that the present invention substantially diverges in design elements and methods from the known art and consequently it is clear that there is a need in the art for an improvement for a pet food supplements. In this regard the instant invention substantially fulfills these needs.

### SUMMARY OF THE INVENTION

In view of the foregoing disadvantages inherent in the known types of pet food supplements now present in the known art, the present invention provides a new pet food supplement wherein the same can be utilized for supplementing the nutritional needs of a pet by administering selected chemicals, vitamins, and minerals, that work in cooperation with each other, for consumption.

It is an objective of the present invention to provide a pet food supplement having vitamins needed for normal cell function, growth, and healthy development of a pet. A vitamin deficiency occurs when you do not get enough of a certain vitamin and a vitamin deficiency can cause health problems. Vitamins can be grouped into two categories: Fat-soluble vitamins, which are stored in the body's fatty tissue, and water-soluble vitamins. Fat-soluble vitamins are best absorbed by the fatty tissue when consumed with fat. Therefore, it is an object of the present invention to provide fatty acids, such as Omega 3 and Omega 6, for simultaneous consumption with the fat-soluble vitamins to increase efficacy of the pet food supplement.

In one embodiment of the present invention, the pet food supplement provides for therapeutic support of joints, immunity, energy, digestion, skin and coat protection, sight, and liver health.

In an alternative embodiment, of the present invention, the pet food supplement comprises effective amounts of turmeric and BIOPERINE® to increase the bioavailability of curcumin shown to play an important role in the prevention and treatment of various pro-inflammatory chronic diseases including neurodegenerative, cardiovascular, pulmonary, metabolic, autoimmune and malignant diseases.

In yet another embodiment of the present invention, the pet food supplement includes glucosamine, chondroitin, Methylsulfonylmethane (MSM), and Sodium Hyaluronate for the maintenance of healthy joints and connective tissues.

In yet another embodiment of the present invention, the pet food supplement provides vitamins paired with minerals and fats that help increase the effectiveness of the vitamins within the body.

It is therefore an object of the present invention to provide a new and improved pet food supplement that has all of the advantages of the known art and none of the disadvantages.

### DETAILED DESCRIPTION OF THE INVENTION

For the purposes of presenting a brief and clear description of the present invention, the preferred embodiment will be discussed as used for mixing with pet food for administration of the pet food supplement.

Reference will now be made in detail to the exemplary embodiment (s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment” does not necessarily refer to the same embodiment.

In one embodiment of the present invention, the pet food supplement provides for the replenishment of nutrients in a pet for therapeutic support of joints, immunity, energy, digestion, skin and coat protection, sight, and liver health. The described pet food supplement discussed herein is designed for an animal weighing 23 kilograms. However, the amount by weight of components in the pet food supplement are configured to be adjusted depending on the weight of an animal consuming the pet food supplement. In one embodiment, the pet food supplement includes, but is not limited to the following therapeutic components: curcumin bioavailability component, joint preserving and building component, fat soluble vitamin absorption component, immune system preserving and building component, and cancer prevention component. In some embodiments, the pet food supplement consists of curcumin bioavailability component, joint preserving and building component, fat soluble vitamin absorption component, immune system preserving and building component, and cancer prevention component.

TABLE 1
Curcumin bioavailability components

| | | |
|---|---|---|
| Turmeric | 150 | mg |
| BIOPERINE ® | 5 | mg |

In the illustrated embodiment, the curcumin bioavailability component includes turmeric and BIOPERINE®, as shown in Table 1. Curcumin has been associated with antioxidant, anti-inflammatory, anticancer, antiviral, and antibacterial activities. BIOPERINE® is an extract from the fruit of black pepper (Piper nigmm) or long pepper (Pigerlongum) that contains 95% of piperine. BIOPERINE® interacts with the curcumin found in the tumeric to increase the relative bioavailability thereof. Additionally, curcumin is shown to play an important role in the prevention and treatment of various pro-inflammatory chronic diseases including neurodegenerative, cardiovascular, pulmonary, metabolic, autoimmune and malignant diseases.

TABLE 2
Joint preserving and building components

| | | |
|---|---|---|
| Glucosamine | 500 | mg |
| Chondroitin Sulfate | 85.5 | mg |
| Methylsulfonylmethane | 350 | mg |
| Sodium Hyaluronate | 6.5 | mg |

In one embodiment of the present invention, the joint preserving and building components of the pet food supplement are designed to maintain and improve joint function. Table 2 provides a list of the joint preserving and building components in one embodiment thereof. The glucosamine in Table 2 includes the following forms, although not limited therewith, glucosamine sulfate, glucosamine hydrochloride, and N-acetyl-glucosamine. Glucosamine is an amino sugar needed to maintain normal joint fluid. Joint fluid surrounds the joints providing them with important nutrients. The joint fluid helps to lubricate and cushion the joints, acting like a shock absorber during movement and insulating the bones from friction. Chondroitin Sulfate delivers nutrients to the joint cartilage, helps to inhibit the enzymes that decompose the joint cartilage and speeds up the formation of a new joint cartilage. Methylsulfonylmethane supplies bioavailable sulfur needed for joints. Sodium Hyaluronate is a joint lubricant and slows down the wear and tear of joints. These ingredients work in synergy to promote joint health and rebuild the joint.

TABLE 3
Fat soluble vitamin absorption components

| | | |
|---|---|---|
| Vitamin A | 0.3 | mg |
| Vitamin D | .000675 | mg |
| Vitamin E | 4.05 | mg |
| Vitamin K | .0006 | mg |
| Omega-3 | 150 | mg |
| Omega-6 | 65 | mg |

In one embodiment of the present invention, the fat-soluble vitamin absorption components, listed in Table 3, of the pet food supplement synergistically provide vitamins and fats, which are critically needed absorption of the vitamins and maintaining overall health. Vitamin A is a group of unsaturated nutritional organic compounds that includes retinol, retinal, retinoic acid, and several provitamin A carotenoids. Vitamin A has multiple functions: it is important for growth and development, for the maintenance of the immune system and good vision. Vitamin D plays an important role in the maintenance of an intact and strong skeleton. Its primary task is to regulate the amount of calcium and phosphorus in the blood by ensuring correct intake from intestines and secretion. Omega 3 and Omega 6 are fatty acids required for normal growth and function of animals.

TABLE 4
Immune system preserving and building components

| | | |
|---|---|---|
| Vitamin B1 | 4 | Mg |
| Vitamin C | 19 | Mg |
| Vitamin E | 4.05 | Mg |
| Manganese | 10 | Mg |
| Zinc | 7 | Mg |

In one embodiment of the present invention, the immune system preserving and building components, listed in Table 4, of the present invention provides vitamins to the cells needed for a healthy immune system. Manganese helps the body utilize a number of vitamins, such as thiamine, and vitamins C and E. Zinc and Vitamin C both boost the immune system, which could be helpful in reducing the negative effects of a multitude of infections. In one embodiment, the Zinc is Zinc Oxide and the Manganese is Manganese Gluconate.

TABLE 5
Cancer prevention components

| | | |
|---|---|---|
| Organic Atlantic Sea Kelp | 300 | mg |
| Grape Seed Extract | 20 | mg |
| Vitamin B6 | 2.8 | mg |
| Vitamin B9 | .005 | mg |
| Vitamin B12 | .0017 | mg |
| Vitamin C | 19 | mg |
| Vitamin E | 4.05 | mg |
| Manganese | 10 | mg |
| Zinc | 7 | mg |

In one embodiment of the present invention, the cancer prevention components, listed in Table 5, of the present invention provide antioxidants to the pet. The organic atlantic sea kelp, grape seed extract, Vitamin C, and Vitamin E are antioxidants that reduce the activity of free radicals. These antioxidants work together to reduce oxidative stress on the cells of the body. This can potentially help detoxify certain toxins and reduce the chance of certain cancers.

TABLE 6
First Embodiment

| | | |
|---|---|---|
| Glucosamine Hydrochloride | 500 | mg |
| Methylsulfonylmethane (MSM) | 350 | mg |
| Chondroitin Sulfate | 85.5 | mg |
| Sodium Hyaluronate | 6.5 | mg |
| Omega 3 Fish Oil | 150 | mg |
| Omega 6 | 65 | mg |
| Turmeric (95% Curcumin) | 150 | mg |
| BIOPERINE ® | 5 | mg |
| Vitamin A | .3 | mg |
| Vitamin B1 | 4 | mg |
| Vitamin B2 | 2.8 | mg |
| Vitamin B3 | 4 | mg |
| Vitamin B5 | 4 | mg |
| Vitamin B6 | 2.8 | mg |
| Vitamin B7 | .008 | mg |
| Vitamin B9 | .005 | mg |
| Vitamin B12 | .0017 | mg |
| Vitamin C | 19 | mg |
| Vitamin D3 | .000675 | mg |
| Vitamin E | 4.05 | mg |
| Vitamin K | .0006 | mg |
| Manganese Gluconate | 10 | mg |
| Zinc Oxide | 7 | mg |
| Organic Atlantic Sea Kelp | 300 | mg |
| Grape Seed Extract | 20 | mg |
| Probiotics and Digestive Enzymes | 20 | mg |
| Liver Flavor | 60 | mg |

In a first embodiment, the pet food supplement comprises the formulation shown in Table 6. In this first embodiment, a key feature of the pet food supplement includes, in combination, (i) a group of compositions for curcumin bioavailability, (ii) a group of compositions for joint preserving and building, (iii) a group of compositions for fat soluble vitamin absorption, (iv) a group of compositions for immune system preserving and building, (v) a group of compositions for cancer prevention, and (vi) the pet food supplement, delivered to the pet as a powder or other chewable medium that can be selectively administered through dosages based on a weight of the pet.

TABLE 7
Second Embodiment

| | | |
|---|---|---|
| Glucosamine Hydrochloride | 500 | mg |
| Methylsulfonylmethane (MSM) | 350 | mg |
| Chondroitin Sulfate | 85.5 | mg |
| Sodium Hyaluronate | 6.5 | mg |
| Omega 3 Fish Oil | 150 | mg |
| Omega 6 | 65 | mg |
| Turmeric (95% Curcumin) | 150 | mg |
| BIOPERINE ® | 5 | mg |
| Vitamin A | .3 | mg |
| Vitamin B1 | 4 | mg |
| Vitamin B2 | 2.8 | mg |
| Vitamin B3 | 4 | mg |
| Vitamin B5 | 4 | mg |
| Vitamin B6 | 2.8 | mg |
| Vitamin B7 | .008 | mg |
| Vitamin B9 | .005 | mg |
| Vitamin B12 | .0017 | mg |
| Vitamin C | 19 | mg |
| Vitamin D3 | .000675 | mg |
| Vitamin E | 4.05 | mg |
| Vitamin K | .0006 | mg |
| Manganese Gluconate | 10 | mg |
| Zinc Oxide | 7 | mg |
| Organic Atlantic Sea Kelp | 300 | mg |
| Grape Seed Extract | 20 | mg |
| Probiotics and Digestive Enzymes | 20 | mg |
| Liver Flavor | 60 | mg |

In an alternative embodiment, the pet food supplement consists of the formulation shown in Table 7.

TABLE 8
Third Embodiment

| | High Range | Low Range | |
|---|---|---|---|
| Glucosamine Hydrochloride | 625 | 375 | mg |
| Methylsulfonylmethane (MSM) | 437.5 | 262.5 | mg |
| Chondroitin Sulfate | 106.875 | 64.125 | mg |
| Sodium Hyaluronate | 8.125 | 4.875 | mg |
| Omega 3 Fish Oil | 187.5 | 112.5 | mg |
| Omega 6 | 81.25 | 48.75 | mg |
| Turmeric (95% Curcumin) | 187.5 | 112.5 | mg |
| BIOPERINE ® | 6.25 | 3.75 | mg |
| Vitamin A | 0.375 | 0.225 | mg |
| Vitamin B1 | 5 | 3 | mg |
| Vitamin B2 | 3.5 | 2.1 | mg |
| Vitamin B3 | 5 | 3 | mg |
| Vitamin B5 | 5 | 3 | mg |
| Vitamin B6 | 3.5 | 2.1 | mg |
| Vitamin B7 | 0.01 | 0.006 | mg |
| Vitamin B9 | 0.00625 | 0.00375 | mg |
| Vitamin B12 | 0.002125 | 0.001275 | mg |
| Vitamin C | 23.75 | 14.25 | mg |
| Vitamin D3 | 0.000844 | 0.000506 | mg |
| Vitamin E | 5.0625 | 3.0375 | mg |
| Vitamin K | 0.00075 | 0.00045 | mg |
| Manganese Gluconate | 12.5 | 7.5 | mg |
| Zinc Oxide | 8.75 | 5.25 | mg |
| Organic Atlantic Sea Kelp | 375 | 225 | mg |
| Grape Seed Extract | 25 | 15 | mg |
| Probiotics and Digestive Enzymes | 25 | 15 | mg |
| Liver Flavor | 75 | 45 | mg |

In an alternative embodiment, the pet food supplement comprises the formulation shown in Table 8. As shown in Table 8, each composition has a range between +/−25% of the mass of each composition found in Table 6. In some embodiments, the therapeutic effects of each composition are less effective outside of the range shown in Table 8.

In one embodiment, the pet food supplement forms a powder that is easily intermixed with wet or dry pet food. In this way, the pet food supplement is consumed by the pet simultaneously with the pet food. Thereby, administration of the present invention to the pet does not require a secondary separate administration, and the pet is cooperative with the process. In another embodiment, the pet food supplement is a solution, pill, and the like. Alternatively, the supplement is administered as a paste, biscuit, jerky treat, chewable flavored tablet adapted to dissolve on the tongue of an animal or admixed with the animal's food. In one embodiment, a single dose of the pet food supplement, the composition of Table 6, is adjusted in accordance with pet having a weight of 23 kg.

It is therefore submitted that the instant invention has been shown and described in what is considered to be the most practical and preferred embodiments. It is recognized, however, that departures may be made within the scope of the invention and that obvious modifications will occur to a person skilled in the art.

Therefore, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A pet supplement for providing curcumin bioavailability, supporting joint health, supporting immunity providing a cancer treatment comprising
a curcumin bioavailability composition comprising:
turmeric and piperine;
a joint preserving and building composition comprising:
glucosamine, Chondroitin Sulfate, Methylsulfonylmethane, and Sodium Hyaluronate;
an immune system preserving and building composition comprising:
Vitamin B1, Vitamin C, Vitamin E, Manganese, and Zinc;
a cancer treatment composition comprising:
kelp, grape seed extract, Vitamin B6, Vitamin B9, Vitamin B12, and the Vitamin C, the Vitamin E, the Manganese, and the Zinc of the immune system preserving and building composition; and
a fat-soluble vitamin absorption composition comprising:
Vitamin A, Vitamin D, Vitamin K, Omega-3, and Omega-6, and the Vitamin E of the immune system preserving and building composition.

2. The pet supplement of claim 1, wherein the turmeric of the curcumin bioavailability composition comprises 150 mg and the piperine of the curcumin bioavailability composition comprises 5 mg.

3. The pet supplement of claim 1, wherein the glucosamine of the joint preserving and building composition comprises 500 mg of Glucosamine Hydrochloride.

4. The pet supplement of claim 1, wherein the joint preserving and building composition comprises 6.5 mg of the Chondroitin Sulfate, 350 mg of the Methylsulfonylmethane, and 6.5 mg of the Sodium Hyaluronate of the joint preserving and building composition.

5. The pet supplement of claim 1, wherein the pet supplement comprises 4 mg of the Vitamin B1, 19 mg of the Vitamin C, 4.05 mg of the Vitamin E, wherein the Manganese comprises 10 mg of Manganese Gluconate, and the Zinc comprises 7 mg of Zinc Oxide.

6. The pet supplement of claim 1, wherein the cancer prevention composition further comprises 300 mg of the kelp, 20 mg of the grape seed extract, 2.6 mg of the Vitamin B6, 0.005 mg of the Vitamin B9, and 0.0017 mg of the Vitamin B12.

7. The pet supplement of claim 1, wherein the fat-soluble vitamin absorption composition further comprises 0.3 mg of the Vitamin A, 0.000675 mg of the Vitamin D, 0.0006 mg of the Vitamin K, 150 mg of the Omega-3, and 65 mg of the Omega-6.

8. The pet supplement of claim 1, further comprising 2.8 mg of Vitamin B2.4 mg of Vitamin B3, 4 mg of Vitamin B5, 0.008 mg of Vitamin B7, 250 mg of Probiotics and Digestive Enzymes, and 60 mg of Liver Flavor.

9. The pet supplement of claim 1, wherein the pet supplement is a powder in an amount targeted for a specific weight of a pet.

10. A pet supplement, consisting of:
a curcumin bioavailability composition consisting of:
turmeric and piperine;
a joint preserving and building composition consisting of:
glucosamine, Chondroitin Sulfate, Methylsulfonylmethane, and Sodium Hyaluronate;
an immune system preserving and building composition comprising:
Vitamin B1, Vitamin C, Vitamin E, Manganese, and Zinc;
a cancer prevention composition consisting of:
kelp, grape seed extract, Vitamin B6, Vitamin B9, Vitamin B12, and the Vitamin C, the Vitamin E, the Manganese, and the Zinc of the immune system preserving and building composition; and
a fat-soluble vitamin absorption composition consisting of:
Vitamin A, Vitamin D, Vitamin K, Omega-3, and Omega-6, and the Vitamin E of the immune system preserving and building composition.

11. A pet supplement adapted to reduce joint inflammation and stiffness in pets and to treat cancer consisting essentially of effective therapeutic amounts by weight of the supplement:
a curcumin bioavailability composition comprising turmeric and piperine, wherein the curcumin bioavailability composition is in an amount of approximately 9.7% by weight;
a joint preserving and building composition comprising glucosamine, Chondroitin Sulfate, Methylsulfonylmethane, and Sodium Hyaluronate, wherein the joint preserving and building composition is in an amount of approximately 53.9% by weight;
an immune system preserving and building composition comprising Vitamin B1, Vitamin C, Vitamin E, Manganese, and Zinc, wherein the immune system preserving and building composition is in an amount of approximately 2.7% by weight;
a cancer treatment composition comprising kelp, grape seed extract, Vitamin B6, Vitamin B9, Vitamin B12, and the Vitamin C, the Vitamin E, the Manganese, and the Zinc of the immune system preserving and building composition, wherein the cancer treatment composition is in an amount of approximately 20.2% by weight;
a fat-soluble vitamin absorption composition comprising Vitamin A, Vitamin D, Vitamin K, Omega-3, and Omega-6, and the Vitamin E of the immune system preserving and building composition, wherein the fat-soluble vitamin absorption composition is in an amount of approximately 13.5% by weight.
